(19) United States
(12) Patent Application Publication
(10) Pub. No.: US 2009/0023181 A1
(43) Pub. Date: Jan. 22, 2009

(54) Method and Device for Detection of Erythromycin-Induced Clindamycin Resistance

(75) Inventors: Katherine S. Sei, El Dorado, CA (US); Gerard D. Bordash, Elk Grove, CA (US); Collette Y. Wehr, El Dorado Hills, CA (US); Barbara L. Zimmer, Davis, CA (US); Judith A. Johnston, Carmicheal, CA (US); Lou Ann Barnett, Sacramento, CA (US)

Correspondence Address: DADE BEHRING INC.;LEGAL DEPARTMENT, 1717 DEERFIELD ROAD, DEERFIELD, IL, 60015, US

(73) Assignee: SIEMENS HEALTHCARE DIAGNOSTICS INC., Deerfield, IL (US)

(21) Appl. No.: 12/060,756

(22) Filed: Apr. 1, 2008

Related U.S. Application Data
(60) Provisional application No. 60/909,663, filed on Apr. 2, 2007.

(51) Int. Cl. C12Q 1/14; C12M 1/34; C12Q 1/04

(52) U.S. Cl. 435/36; 435/288.4; 435/34

(57) ABSTRACT

A device and method are provided for determination of erythromycin-induced clindamycin resistance. The device is preferably a test panel containing one or more wells each having a known concentration(s) of erythromycin and known concentration(s) of clindamycin. After inoculation of the wells with a sample and incubation, the wells are analyzed for growth and to determine whether sample contains a microorganism that expresses erythromycin-induced clindamycin resistance.

This application claims priority to U.S. Provisional Application No. 60/909,663, filed Apr. 2, 2007.

### FIELD OF THE INVENTION

The present invention relates to methods and devices useful for the determination of erythromycin-induced resistance to clindamycin. The methods and devices of the present invention may be combined anti-microbial susceptibility testing and microorganism identification devices.

### BACKGROUND OF THE INVENTION

The field of clinical microbiology and the treatment and management of infectious diseases has progressed dramatically over the last several decades. However, life threatening and debilitating systemic and localized microbial infections remain a major healthcare problem Mortality resulting from infectious agents remains particularly high among infants, the elderly, the immuno-suppressed, patients in long-term care facilities, and nursing homes. Moreover, the emergence of multi-drug resistance organisms have increased the challenges of caring for hospitalized patients. Hospital acquired infections (nosocomial infections) caused by drug resistant organisms add significantly to patient suffering, increased hospital stays, iatrogenic mortality, and increased healthcare costs.

Inadequately or improperly treated microbial infections are largely responsible for the rise of multiple drug resistant strains of bacteria that cause many nosocomial infections. Drug resistance, specifically antibiotic resistance, often occurs when the antibiotic used to treat an infection is either improperly selected, prescribed in a fashion that does not effectively eradicate the infectious agent, or as a result of poor patient compliance. Furthermore, when ineffective or unnecessary antibiotics are prescribed, any infecting bacteria present continues to multiply unabated, often resulting in life threatening complications necessitating expensive, aggressive treatments. Therefore, the accurate and rapid diagnosis of a potential infectious agent is critical to improved patient care, reduced healthcare costs and the preservation of antimicrobial efficacy.

Inducible clindamycin resistance in Staphylococcus species has become an important clinical issue. Macrolide resistance in staphylococci may be due to ribosomal target modification that affects the activities of both macrolides and clindamycin, called macrolide-lincosamide-streptogramin B (MLS_{B}) resistance, which can be induced by the genes erm(A) or erm(C). Certain strains of Staphylococcus exhibit constitutive resistance to clindamycin but others only exhibit resistance after induction by erythromycin. While strains that demonstrate constitutive resistance to clindamycin can normally be detected by standard susceptibility testing methods, inducible resistance (MLS_{B}i) present in some strains is not routinely detected by standard broth- or agar-based susceptibility test methods. It is important to distinguish the MLS_{B}i strains from macrolide-resistant strains that contain the gene msr(A), encoding an efflux pump that affects only macrolides, not clindamycin.

The goal of routine detection of clindamycin resistance among clinically significant staphylococcal isolates is twofold. First, prior investigations have demonstrated the potential for clinical failures when patients infected with MLS_{B}i strains are treated with clindamycin for various types of infections. However, to regard all macrolide-resistant staphylococci as clindamycin resistant would deny potentially safe and effective therapy for patients infected with isolates that carry only the macrolide efflux mechanism. The percentage of clinical staphylococcal isolates that demonstrate macrolide efflux compared to MLS_{B} resistance varies widely by geographic location or patient group. Therefore a second benefit of routine testing for inducible clindamycin resistance is to clearly identify those strains that remain susceptible to clindamycin despite macrolide resistance. For these reasons, routine testing of staphylococcal isolates for inducible clindamycin resistance is advocated by the Clinical Laboratory Standards Institute (CLSI), formerly known as the National Committee for Clinical Laboratory Standards (NCCLS).

Public health experts are urging clinicians and physicians to prescribe more narrow-spectrum antibiotics in order to slow the spread of resistance among bacterial strains. But to do so, the identity and antibiotic susceptibility of the microorganism must be determined expeditiously. Standard culture based methods can take up to weeks to provide this information to clinicians Antibiotic misuse most often occurs as a result of inadequate information concerning the causative pathogen rather than from inappropriate prescribing behavior. Clinical laboratories therefore will have a great advantage if they have automated systems that can identify the infectious agent and its potential resistance in hours rather than days.

Therefore, devices and methods for the rapid and accurate determination of erythromycin-induced resistance to clindamycin in patient isolates are needed to improve treatment of infectious disease and decrease antibiotic resistance.

### SUMMARY OF THE INVENTION

The present invention provides methods and devices for the automated rapid and accurate determination of erythromycin-induced resistance to clindamycin in patient isolates.

In one aspect of the invention, a device for use in the determination of erythromycin-induced clindamycin resistance having a panel having one or more wells containing a growth media, a known amount of erythromycin, and a known amount of clindamycin.

In another aspect of the invention, a method is provided for the determination of erythromycin-induced clindamycin resistance. The method includes providing a panel having one or more wells containing a growth media, a known amount of erythromycin, and a known amount of clindamycin; inoculating the one or more wells with a sample suspected of containing microorganisms; incubating the panel at conditions suitable for growth of the microorganisms in the wells; analyzing the one or more for growth of the microorganisms; and determining the presence or absence of erythromycin-induced clindamycin resistance based on the growth of microorganisms in the wells.

Other aspects of the invention will become apparent to those skilled in the art based on the description that follows.

### BRIEF DESCRIPTION OF THE DRAWINGS

FIG. 1 illustrates a plan view of a device for determining the presence or absence of erythromycin-induced resistance to clindamycin according to one aspect of the invention.

### DETAILED DESCRIPTION OF THE INVENTION

The present invention provides methods and devices for the determination of erythromycin-induced resistance to clindamycin in patient isolates.

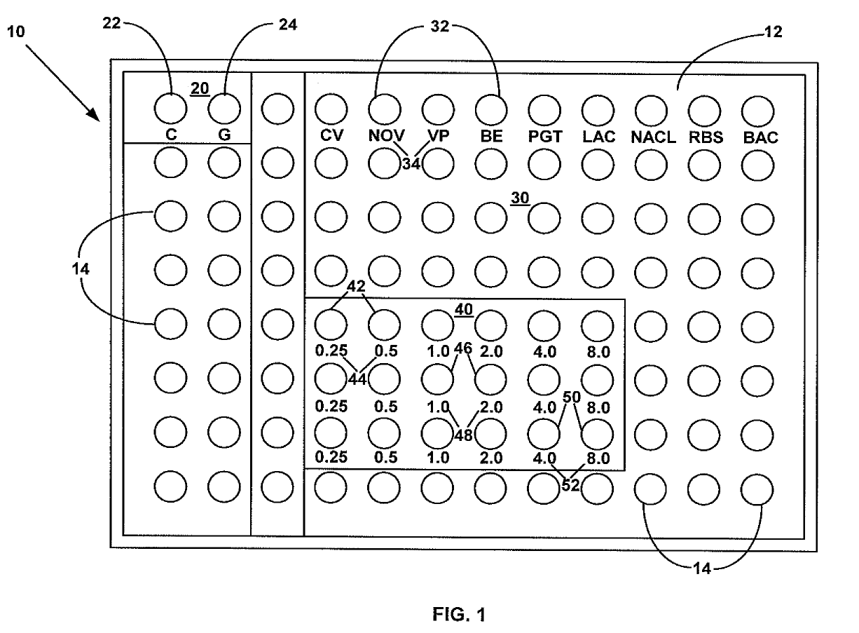

According to one aspect of the present invention, a device for use in the determination of erythromycin-induced clindamycin resistance is shown generally at FIG. 1. In this embodiment, the device is shown in the form of a test panel 10 having a generally planar surface 12 with a plurality of reaction chambers (or wells 14). The wells 14 are shown arranged in a rectangular configuration in an array of twelve wells across and eight wells down, for a total of ninety-six wells, however it should be understood that the well panels of the present invention are not limited to this configuration.

The test panel 10 is divided into a plurality of sub-sections including the control section 20, the microbial identification (ID) section 30, and the antimicrobial susceptibility testing (AST) section 40. Those skilled in the art will understand that the panel may include other sub-sections which are not necessary to describe the present invention. The control section 20 includes a control well 22 and a growth well 24. The growth well 24 contains a growth medium for the determination of whether a particular organism can grow in the panel. The growth medium 24 also shows that the panel has been inoculated. A panel is not read if there is no growth in the growth well. The control well 22 is not inoculated and will show growth only if the panel is contaminated. A panel is not read if there is growth in the control well.

The microbial identification section 30 includes a plurality of ID wells 32, each containing a growth medium 24 and a single antimicrobial compound identified by indicia 34. Each of the ID wells 32 is inoculated and will only show growth if the microbes in the sample are not resistant to the particular antimicrobial compound in each well. The aggregate of these susceptibility results can be used in an algorithm to identify the microbes in the sample. Such algorithms are well-known in the art and need not be discussed. Those skilled in the art will recognize antimicrobial compounds may be included in this section and those disclosed here are exemplary only.

The antimicrobial susceptibility testing portion 40 of the exemplary test panel 10 shown includes three series or sets of wells 42, 46, and 50, each containing six wells, for antimicrobial susceptibility testing (AST). Those skilled in the art will recognize that other embodiments of the present invention may include more or fewer series of wells, and more or fewer number of wells in each set. The first set of wells 42 each contain a growth medium and a known amount of erythromycin. The known amount of erythromycin is different for each well in the series, and is indicated by indicia 44 (shown here in the units μg/ml). Preferably, the wells 42 each exclude clindamycin. The second set of wells 46 each contain a growth medium and a known amount of clindamycin. The known amount of clindamycin is different for each well in the series, and is indicated by indicia 48 (shown here in the units μg/ml). Preferably, the wells 46 each exclude erythromycin. The third set of wells 50 each contain a growth medium, known amount of erythromycin and a known amount of clindamycin. The known amount of clindamycin is different for each of the wells 50 and is indicated by indicia 52 (shown here in the units μg/ml). The known amount of erythromycin in each well 50 may be constant or may be different for each well. In one embodiment, the known amount of erythromycin is less than the known amount of clindamycin in each well 50.

Those skilled in the art will recognize the various concentrations that may be used in each of the wells 42, 46, and 50. Typically, the wells 42 will contain a known amount of erythromycin in the range of about 0.25 μg/ml to about 16.0 μg/ml. The wells 46 will typically contain about 0.25 μg/ml to about 16.0 μg/ml of clindamycin. The wells 50 will typically contain about 0.25 μg/ml to about 16.0 μg/ml of clindamycin, and from about 0.1 μg/ml to about 8.0 μg/ml of erythromycin.

The test panel is preferably used in a method for the determination of erythromycin-induced clindamycin resistance according to another aspect of the invention. The method includes inoculating the panel 10 with a sample suspected of containing microorganisms; incubating the panel at conditions suitable for growth of the microorganisms in the wells 42, 46, and 50; analyzing the wells 42, 46, 50 for growth of the microorganisms; and determining the presence or absence of erythromycin-induced clindamycin resistance based on the growth of microorganisms in the wells 42, 46, and 50.

If a microorganism grows in the first set of the wells 42, or in enough of the set of wells 42 such that its minimum inhibitory concentration is greater than a threshold (i.e., MIC>4 μg/ml) the microorganism is said to be erythromycin resistant. If an such erythromycin-resistant isolate does not grow in wells 46 containing clindamycin alone, or does not grow in most of them (i.e., MIC</=0.5 μg/ml), then the microorganism is said to be clindamycin-susceptible. Then, if the microorganism grows in wells 50, or in enough of the wells such that its minimum inhibitory concentration is greater than a threshold (i.e., MIC>2 μg/ml), the isolate is reported as having inducible resistance to clindamycin. If the isolate does not grow in the combination of erythromycin and clindamycin (wells 50), it is reported as being susceptible to clindamycin, i.e., contains the msrA gene. The threshold may be set according to well-known procedures in the art.

It should be noted that in one embodiment of the invention, where a microorganism is already known to be erythromycin resistant and clindamycin-susceptible, only the third series of wells 50 is needed to determine whether the microorganism has erythromycin induced resistance to clindamycin.

This present invention precludes having to perform additional tests for the erm or msr genes or having to perform disk diffusion tests with erythromycin and clindamycin disks in additional to performing the routine MIC tests.

The wells of the test panels can be read manually or as part of an automated system. An exemplary automated assay system is the WALKAWAY® system available from Dade Behring Inc, MicroScan® Systems (West Sacramento, Calif.). The WALKAWAY® system is an automated system which includes microtiter identification and antimicrobial susceptibility testing panels, interprets biochemical results through use of a photometric or fluorogenic reader and generates results that can be interfaced with hospital mainframe information systems. Several embodiments of WALKAWAY® system and components thereof are disclosed in U.S. Pat. Nos. 5,888,760, 5,645,800, 5,518,686, 4,676,951, 4,643,879, 4,681,741 and 4,448,534 and co-pending U.S. patent application Ser. No. 10/967,062 filed Oct. 15, 2004. It should be understood, however, that the methods of the present invention may readily be adapted to other systems as well.

The erythromycin-induced clindamycin resistance wells may be included in a standard semi-automated antimicrobial susceptibility test (AST) panel, such as a MicroScan® AST panel, and provide results at the same time as minimum inhibitory concentration (MIC) values for erythromycin and clindamycin are available.

The antimicrobial susceptibility tests are variations and miniaturizations of the broth dilution susceptibility tests. In one embodiment of the present invention, various antimicrobial agents are serially diluted in autoclaved distilled water to concentrations bridging the range of clinical interest. After inoculation with a standardized suspension of organism in a variation (modification) of Mueller-Hinton broth and incubation at 35° C. in the WALKAWAY® System for 3.5 to 24 hours.

The susceptibility portion of the panel is read spectrophotometrically using the calorimeter of an appropriate automated assay device (e.g. the WALKAWAY®) using one or more distinct wavelengths of light, e.g. 405, 505 and 620 nanometers (nm), at specified times from 30 minutes to 24 hours. The times and wavelengths described herein are exemplary and not limiting, however. The read times and/or the number and type of wavelength used may readily be adjusted to enhance the accuracy and rapidity of the determinations.

After turbidity has been read, the raw data is processed on board the assay system. At each time point, specific questions are asked of the raw data in the related software (e.g. MicroScan® Data Management System software, Dade Behring Inc., West Sacramento, Calif.) to determine if further incubation is required or whether the susceptibility results can be reported. For example, at the 6.5 hour read, data from the 4.5 and 5.5 hour reads are used to determine if a MIC can be determined or if further incubation is required.

It should be readily understood by those persons skilled in the art that the present invention is susceptible of a broad utility and application. Many embodiments and adaptations of the present invention other than those herein described, as well as many variations, modifications and equivalent arrangements will be apparent from or reasonably suggested by the present invention and the foregoing description thereof, without departing from the substance or scope of the present invention.

Accordingly, while the present invention has been described herein in detail in relation to specific embodiments, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements, the present invention being limited only by the claims appended hereto and the equivalents thereof.

We claim:

1. A device for use in the determination of erythromycin-induced clindamycin resistance comprising:
a panel having a first at least one well containing a growth medium, a known amount of erythromycin, and a known amount of clindamycin.

2. The device according to claim 1, wherein the panel further comprises a second at least one well containing a growth medium and a known amount clindamycin.

3. The device according to claim 1, wherein said second at least one well excludes an antimicrobial effective amount of erythromycin.

4. The device of claim 1, wherein the known amount of erythromycin in said first at least one well is less than the known amount of clindamycin in said first at least one well.

5. The device of claim 1, wherein said first at least one well comprises a first plurality of wells, each well of the first plurality having a different known amount of clindamycin.

6. The device of claim 2, wherein said second at least one well comprises a second plurality of wells, each well of the second plurality having a different known amount of clindamycin.

7. The device of claim 3, further comprising a third at least one well containing a growth medium and a known amount of erythromycin,

8. The device of claim 7, wherein said third at least one well excludes an antimicrobial effective amount of clindamycin.

9. The device of claim 7, wherein said third at least one well comprises a third plurality of wells, each well of the third plurality having a different known amount of erythromycin.

10. A method for the determination of erythromycin-induced clindamycin resistance comprising:
providing a panel having at least one well containing a growth media, a known amount of erythromycin, and a known amount of clindamycin;
inoculating said at least one well with a sample suspected of containing microorganisms;
incubating said panel at conditions suitable for growth of the microorganisms in the wells;
analyzing the at least one well for growth of the microorganisms;
determining the presence or absence of erythromycin-induced clindamycin resistance based on the growth of microorganisms in said at least one well.

11. The device of claim 10, wherein the known amount of erythromycin in said at least one well is less than the known amount of clindamycin in said at least one well.

12. The device of claim 10, wherein said first at least one well comprises a plurality of wells, each well of the plurality having a different known amount of clindamycin.

13. The method of claim 10, wherein the microorganism is a strain of Staphylococcus.

14. A method for the determination of erythromycin-induced clindamycin resistance comprising:
providing a panel having a first at least one well containing a growth media, a known amount of erythromycin, and a known amount of clindamycin and a second at least one well containing a growth media and a known amount of clindamycin;
inoculating said first at least one well and said second at least one well with a sample suspected of containing microorganisms;
incubating said panel at conditions suitable for growth of the microorganisms in the wells;
analyzing said first at least one well and said second at least one well for growth of the microorganisms;
determining the presence or absence of erythromycin-induced clindamycin resistance based on the growth of microorganisms in said first at least one well and said second well.

15. The device according to claim 14, wherein said second at least one well excludes an antimicrobial effective amount of erythromycin.

16. The device of claim 14, wherein said second at least one well comprises a second plurality of wells, each well of the second plurality having a different known amount of clindamycin.
